(19) United States
(12) Patent Application Publication
(10) Pub. No.: US 2022/0406455 A1
(43) Pub. Date: Dec. 22, 2022

(54) MEDICAL KIOSK

(71) Applicant: HealthStation, INC., Dysart, IA (US)

(72) Inventors: Rick TRESNAK, Dysart, IA (US); Jonathan LARA, Ankeny, IA (US)

(73) Assignee: HealthStation, INC., Dysart, IA (US)

(21) Appl. No.: 17/844,485

(22) Filed: Jun. 20, 2022

Related U.S. Application Data
(60) Provisional application No. 63/212,701, filed on Jun. 20, 2021.

(51) Int. Cl. G16H 40/67; G16H 50/20; G06Q 20/40

(52) U.S. Cl. CPC G16H 40/67; G16H 50/20; G06Q 20/4097

(57) ABSTRACT

A medical kiosk has a frame that encloses a medical diagnosis chair and a video screen within is configured for interactive video and audio communication between a medical provider and a user. A financial transaction interface is configured to provide secure payment processing that allows the user to pay and enter the medical kiosk. A UV light disinfection system is disposed within said frame to provide a sterile decontaminated user space. The medical kiosk includes a network communication module and a system control unit in electrical communication with the medical diagnosis chair, the video screen, the financial transaction interface, the UV light disinfection systems and the network communication module wherein the user is provided with non-invasive medical conferencing with the medical provider to remotely diagnose and treat the user.

### CROSS REFERENCE TO RELATED APPLICATIONS

This application claims priority and herein incorporates by reference U.S. provisional patent application 63/212,701, filed Jun. 20, 2021.

### BACKGROUND OF THE INVENTION

Medical services are traditionally provided to individuals at a doctor's office or medical facility. Typically, an individual contacts his/her medical provider when the individual requires some type of medical assistance. The medical provider then sets an appointment time and date for the individual to see the medical provider. Many times, the time and date of the appointment are inconvenient for the individual. Furthermore, the individual seeking medical assistance may desire or need more immediate medical assistance and cannot wait for the time and date set by the medical provider. In such situations, the individual goes to the emergency room of a hospital or some type of medical clinic (e.g., Minute Clinic, Take Care Clinic, Urgent Care Clinic, etc.), assuming that such clinics are available or convenient to visit. In other situations, especially in smaller towns and rural areas, a medical facility may be located many miles away thus making a visit to such a facility very inconvenient. As such, many individuals who should seek medical care decide to not seek the advice of medical personnel due to the inconvenience of having to travel large distances to a medical facility.

### SUMMARY OF THE INVENTION

A medical kiosk has a frame that encloses a medical diagnosis chair and a video screen within is configured for interactive video and audio communication between a medical provider and a user. A financial transaction interface is configured to provide secure payment processing that allows the user to pay and enter the medical kiosk. A UV light disinfection system is disposed within said frame to provide a sterile decontaminated user space. The medical kiosk includes a network communication module and a system control unit in electrical communication with the medical diagnosis chair, the video screen, the financial transaction interface, the UV light disinfection systems and the network communication module wherein the user is provided with non-invasive medical conferencing with the medical provider to remotely diagnose and treat the user.

Other features and advantages of the instant invention will become apparent from the following description of the invention which refers to the accompanying drawings.

### BRIEF DESCRIPTION OF THE DRAWINGS

FIG. 1 is a perspective drawing of a medical kiosk according to an embodiment of the invention.

FIG. 2 is a see-through view of the medical kiosk shown in FIG. 1.

FIG. 3 is a system diagram of a network enabled embodiment of a plurality of medical kiosks according to an embodiment of the invention.

FIG. 4 is a system diagram of a satellite network enabled embodiment of the invention.

FIG. 5 is a system diagram of a hybrid network enabled embodiment of the invention.

FIG. 6 is a perspective drawing of the medical kiosk shown in FIG. 1 with an accompanying medical dispensary unit according to an embodiment of the invention.

FIG. 7 is a perspective drawing of the medical kiosk shown in FIG. 1 with a backup generator.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

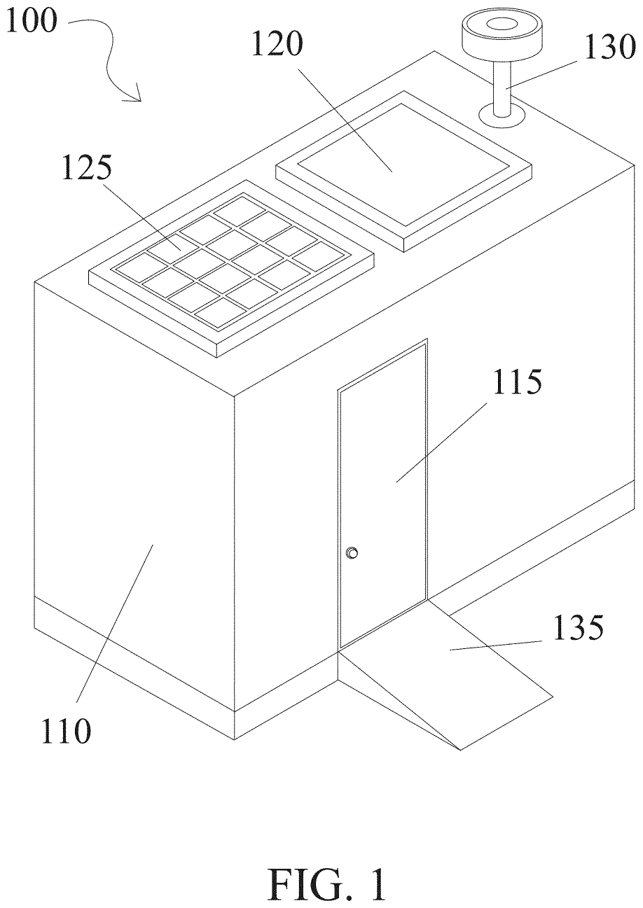

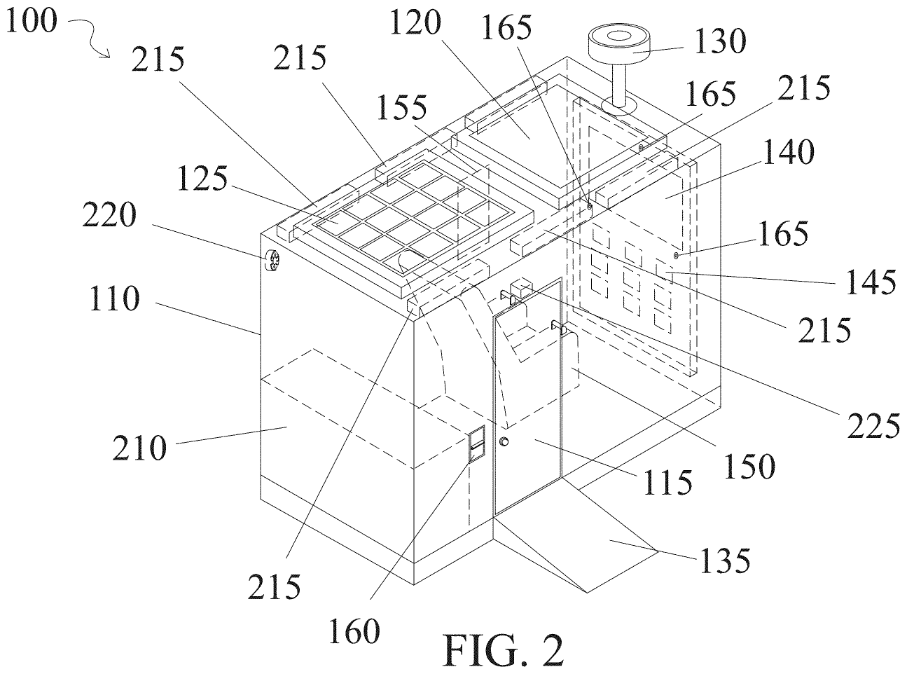

Referring to FIGS. 1 and 2, a medical kiosk 100 is shown having a frame 110 with a door 115 and a ramp 135. A skylight 120 is disposed in a top portion of frame 110 to reduce a user's stress level. A solar array 125 is shown to generate electrical energy. A communication antenna 130 is provided to connect medical kiosk 100 to a communication network.

Within medical kiosk 100 a medical diagnosis chair 150 has various sensors such as, but not limited to, a scale to measure the weight of a user, pulse and oxygen blood level sensors, etc. as is known in the art. The medical diagnosis sensors are non-invasive. A video screen 140 is provided to allow the user to interact with a consulting medical professional. A communication antenna 130 is provided to allow connection to a network. A system control unit 210 provides all computing processes, communication module, and payment processing functions required to operate. A card reader 160 is provided to open medical kiosk 100 and to operate a “Point-of-Sale” system that authorizes payment for all services rendered as well as provide for any incidental damage that a user might cause. Card reader 160 accepts a credit card (not shown) but could also read other information such as insurance card information. A locking mechanism is configured to respond to card reader 160 to secure access to medical kiosk 100. Of course any secure financial transaction interface may be used such as, but not limited to, “tap-to-pay” chip based hardware or bio-metric authorization technologies. Card reader 160 is in electrical communication with a system control unit 210 to authorize and validate a user's credit card. System control unit 210 utilizes a computing device with internet access that is linked to a credit card processing service as is known in the art.

The attending medical professional is shown on video screen 140 and the user's image and voice is transmitted to the medical professional by at least one video camera 165. Video camera 165 is also used by the medical professional to view particular areas of concern during a diagnosis. Multiple video cameras 165 may be used to further enhance the diagnosis information available to the treating medical professional. Information or interactive instructions may be presented or responded to through a touch screen display 145 where the user can input answers or queries in addition to voice communication. AIso provided is all the electrical wiring (not shown) required to connect all necessary equipment and communication software and hardware as is known in the art.

A smoke and carbon monoxide detector 220 is provided to ensure safe levels of atmosphere within medical kiosk 100. Additional safety equipment, such as, but not limited to fire extinguishers, fire suppression system, oxygen tank, Automated External Defibrillator (AED) etc. may be provided to further enhance user safety. A medical equipment compartment 155 is provided to hold various medical equipment such as, but not limited to, blood pressure cuffs or other medical non-invasive diagnosis equipment as is known in the art. Instructions on when and how to use the equipment may be provided through written instructions appearing on video screen 140 or touch screen 145, and/ or voice instructions provided by medical provider 170.

A motion detector 225 is provided to determine when a user is present and when the user has left. Once medical kiosk is empty, door 115 is locked and a plurality of UV sterilization lights 215 energize to disinfect the entire space and equipment within without the need for any human intervention. A disinfectant routine is run by system control unit 210 and once the disinfection routine is complete, medical kiosk 100 is ready for a new user. Medical kiosk 100 is configured to be fully compliant with the American with Disabilities Act (ADA). An accessibility ramp 135 is provided to facilitate users with ambulatory assistive devices. Additionally, the interior design is configured to fall within the guidelines outlined in the ADA. The interior surface of medical kiosk 100 is painted with antimicrobial paint or antimicrobial treatments to further protect the user from the spread of illness or disease. Any suitable antimicrobial/antiviral paint may be used as is known in the art. Other antimicrobial options can include, but not limited to copper or silver coatings, etc. as is known in the art.

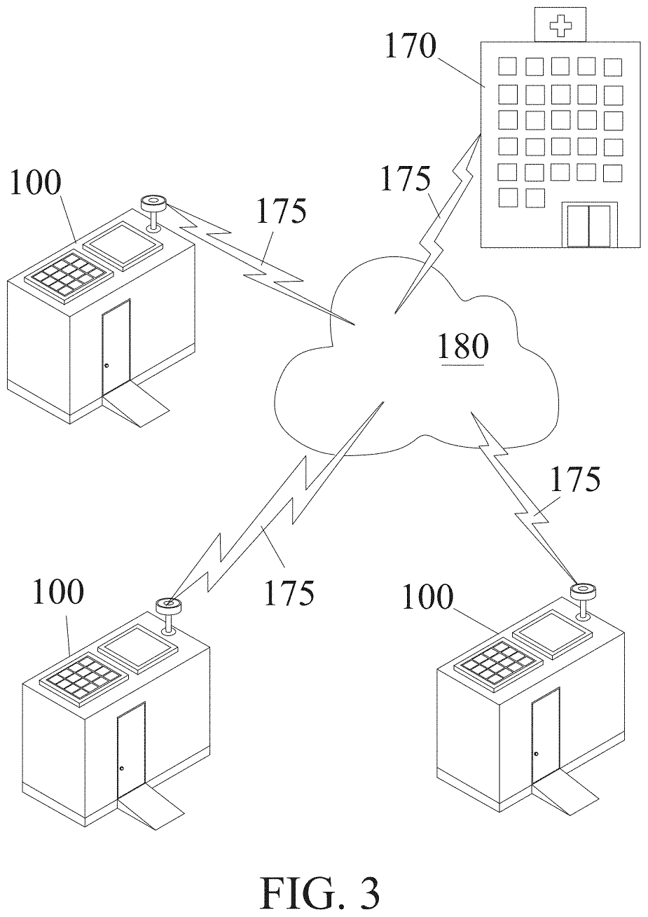

Referring now to FIGS. 3-5, various communication networks embodiments are shown to allow medical kiosks 100 to communicate with medical providers 170 to provide real-time tele-medicine to users. FIG. 3 shows an internet communication network 180 where medical providers 170 are connected to each medical kiosk 100 by connecting through an internet connection 175. Typical security protocols are used to maintain patient confidentiality and financial security as is known in the art.

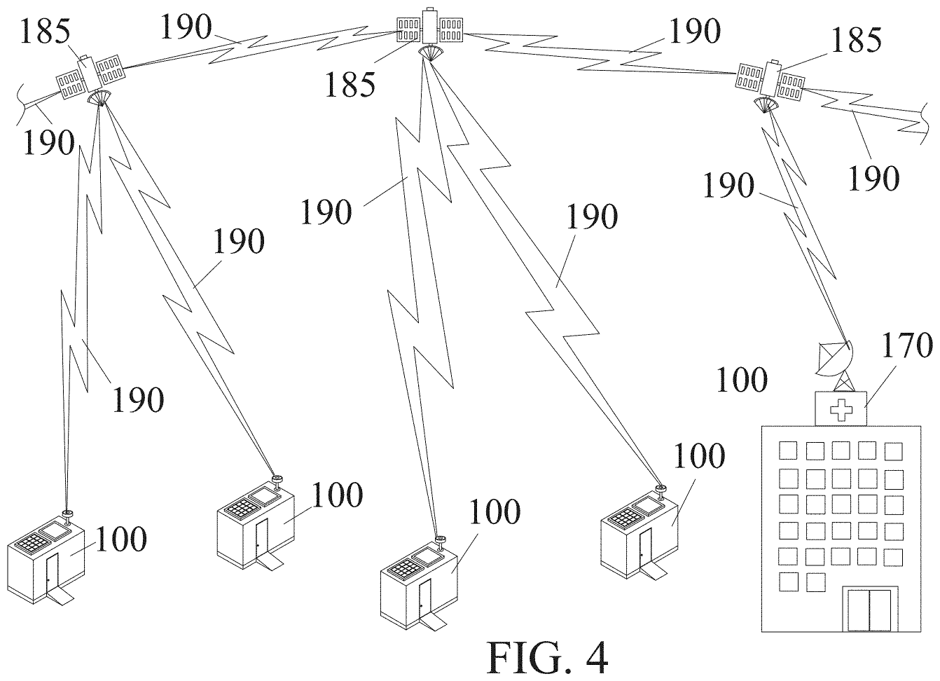

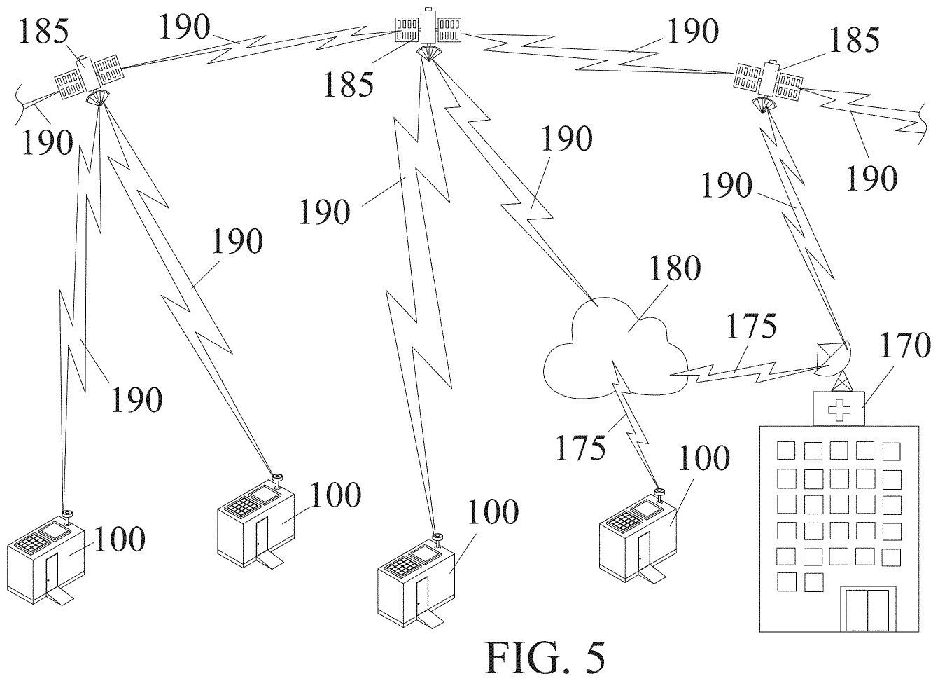

FIG. 4 illustrates a satellite communication embodiment using secure communication protocols to link medical kiosk 100 to satellites 185 and medical provider 170 through a satellite connection 190. This embodiment is typically more secure than standard internet networks. FIG. 5 illustrates a hybrid embodiment that utilizes both satellite communications as well as internet connections. Of course, any suitable communication network may be used such as mobile 5G networks, LAN, MAN, and WAN, etc. as is known in the art. Patient confidentiality and secure financial transactions must be implemented regardless of the communication network used.

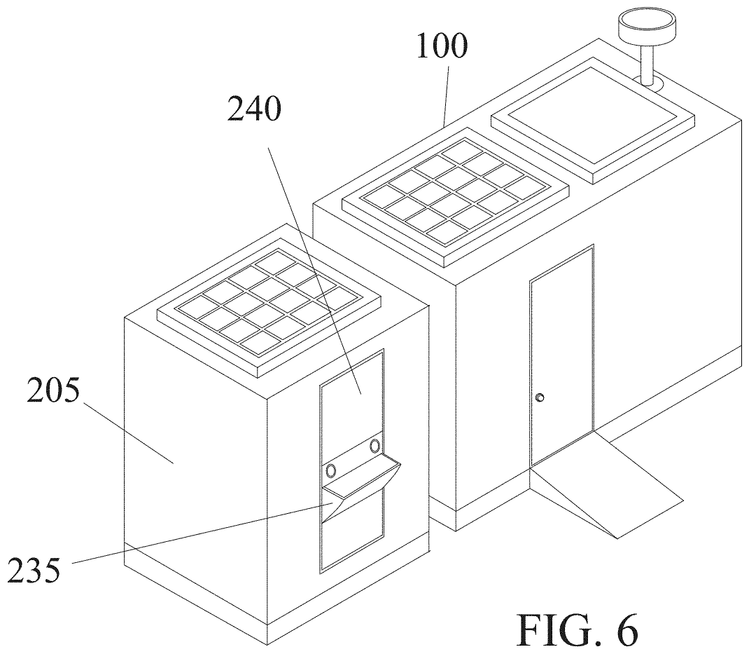

FIG. 6 shows a medical kiosk 100 connected to a medical dispensary unit 205. Medical dispensary unit 205 is configured to dispense medical prescriptions and medical equipment such as, but not limited to, orthopedic braces, nebulizers, Holter monitors, urine cups, CLIA waived tests and send-out tests. Medical dispensary unit 205 is automated using mechanical and electronic components to dispense the desired medication and equipment. Prescriptions are logged and configured to meet all federal and state medical requirements for prescriptions. Various security protocols including biometric confirmation may be used to verify the user's identity as is known in the art. The dispensed material may be accessed through a dispensing slot 235 or a door (not shown) once authorized by system control unit 210. A video screen 240 may be provided to aid the user in using medical dispensary unit 205. Medical dispensary unit 205 may be powered using solar panels, electrical connection with medical kiosk 100, or any other satisfactory power source.

In embodiments not including medical dispensary unit 205 or if additional medical supply is needed, medications or medical supplies may be ordered and delivered to the user, or a prescription can be delivered to a pharmacy of the user's choice.

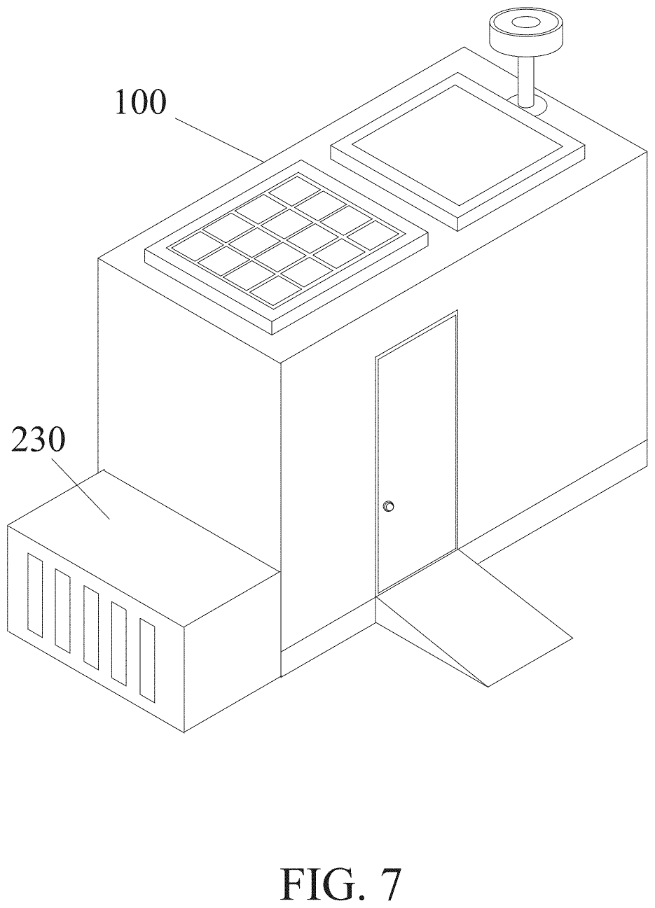

FIG. 7 illustrates an embodiment that utilizes a generator 230 to either provide the required electrical power or to provide a backup source of electrical energy if the required energy supplied by solar panels 125 is insufficient. Of course, medical kiosk 100 is also able to be powered through connection to a standard electrical connection as is known in the art. AIthough not shown, all required wiring and/or wireless connections to electrically connect all the various components of medical kiosk 100 is provided as is known in the art. Wiring can be provided within an interior wall if one is used or may be enclosed within tubing or channels mounted therein as is known in the art. Wireless connections such as, but not limited to BLUETOOTH, WiFi, IR, RF, or other suitable wireless technology may be used to electrically connect various components to system control unit 210 as is known in the art.

To construct medical kiosk 100, shipping containers may be used and repurposed to provide frame 110. Of course frame 110 may be made of any suitable material including, but not limited to, steel, aluminum, carbon fiber, etc. as long as it is durable and resistant to damage or break-in.

Medical kiosk utilizes an Application Programming Interface (API) for data transfer between user and medical provider 170. A patient health record software/database is provided to allow user to conveniently access their medical records as is known in the art. A mobile app may be used to provide user access. System control unit 210 utilizes a computer that runs an Artificial Intelligence (AI) program that can help the user understand the instructions necessary for medical provider 170 to diagnosis and deliver medical advice. The AI can also be used to detect the level of user stress, determine depression and other subtle indications that help diagnose the issue and put the user at ease. Medical scanning is utilized to help diagnose issues and provide medical provider 170 with the information needed and may utilize a range of non-invasive medical scanning procedures as is known in the art.

Each time a user leaves medical kiosk 100, an antimicrobial and antiviral disinfectant routine is run utilizing UV lights 215. Additionally, medical kiosk 100 utilizes a HVAC system with HEPA filtration (not shown) as is known in the art. AIl or some parts of the software run by system control unit 210 may be custom developed or utilize off-the- shelf software to provide the necessary programming. System control unit 210 also controls all financial authorizations, disinfectant routines, locking and unlocking functions, emergency notification in the event of a user emergency during use, ventilation and power control, medical scans, network links and communications, and security. System control unit 210 also utilizes communication hardware and software required for network communication. Medical kiosk 100 is modular in configuration and can be placed either in an outdoor or indoor location.

AIthough the instant invention has been described in relation to particular embodiments thereof, many other variations and modifications and other uses will become apparent to those skilled in the art.

What is claimed is:

1. A medical kiosk comprising:
a frame;
said frame having four walls, a floor and a ceiling;
a door disposed in one of said wall of said frame;
a medical diagnosis chair disposed within said frame;
a video screen configured for interactive video and audio communication disposed in front of said medical diagnosis chair;
a financial transaction interface configured to provide secure payment processing;
a UV light disinfection system contained with said frame;
a network communication module; and
a system control unit in electrical communication with said medical diagnosis chair, said video screen, said financial transaction interface, UV light disinfection systems and said network communication module.

2. The medical kiosk according to claim 1 further comprising a motion detector in electrical communication with said system control unit wherein a disinfection routine is run in response to a programmed instruction set when a selected motion control criteria is met.

3. The medical kiosk according to claim 1 further comprising a communication antenna attached to an exterior portion of said frame wherein said communication antenna is in electrical communication with said system control unit.

4. The medical kiosk according to claim 1 wherein said financial transaction interface is a credit card scanner.

5. The medical kiosk according to claim 1 further comprising at least one medical scanner disposed within said frame and further configured to scan a user seated in said medical diagnosis chair and electrically transmit scan data to said system control unit.

6. The medical kiosk according to claim 5 wherein said system control unit comprises a central processing unit, random access memory (RAM), read-write mass storage device, input and output interface and communication hardware necessary to communicate data over a network.

7. The medical kiosk according to claim 6 wherein said control system is configured to run an artificial intelligence routine to help diagnose medical conditions of a user seated in said medical diagnosis chair.

8. The medical kiosk according to claim 6 wherein said system control unit is configured to implement secure two-way communication between a selected medical provider and said user.

9. The medical kiosk according to claim 1 further comprising a touch screen disposed within reach of said medical diagnosis chair where a user can make selections and answer queries using said touch screen.

10. The medical kiosk according to claim 1 further comprising a compartment disposed within said frame configured to hold medical equipment.

11. The medical kiosk according to claim 1 further comprising an environmental detector mounted within said frame wherein air quality conditions within said medical kiosk is monitored.

12. The medical kiosk according to claim 11 wherein said environmental detector is a smoke and CO detector.

13. The medical kiosk according to claim 1 wherein said frame is made of a storage container.

14. The medical kiosk according to claim 1 wherein at least some of an interior portion of said medical kiosk is coated with an antimicrobial/antiviral paint.

15. The medical kiosk according to claim 8 further comprising a medical dispensary unit in electrical communication with said system control unit wherein medical supplies are dispensed when authorized.

16. The medical kiosk according to claim 1 further comprising a generator electrically connected to said medical kiosk to provide electrical power.

17. The medical kiosk according to claim 1 further comprising a solar panel array disposed on an exterior upper surface of said frame electrically connected to said medical kiosk to provide at least some electrical power to said medical kiosk.

18. The medical kiosk according to claim 1 further comprising an accessibility ramp attached to a bottom portion of said door whereby accessibility to said medical kiosk is enhanced.

19. The medical kiosk according to claim 4 wherein access to said kiosk through said door is gained by an authorized transaction made in response to a payment request initiated using said credit card scanner.

20. The medical kiosk according to claim 1 further comprising a skylight disposed in a ceiling portion of said frame.
